(19) United States
(12) Patent Application Publication
(10) Pub. No.: US 2009/0069802 A1
(43) Pub. Date: Mar. 12, 2009

(54) ELECTROSURGICAL ELECTRODE AND METHOD FOR USE

(76) Inventors: Jon C. Garito, Oceanside, NY (US); Alan G. Ellman, Hewlett, NY (US)

Correspondence Address: DARBY & DARBY P.C., P.O. BOX 770, Church Street Station, New York, NY, 10008-0770, US

(21) Appl. No.: 11/851,251

(22) Filed: Sep. 6, 2007

(51) Int. Cl. A61B 18/14

(52) U.S. Cl. 606/37

(57) ABSTRACT

An electrosurgical electrode for use in a surgical procedure includes an elongated body in the shape of a Freer elevator having a working end portion in the shape of a spoon at a distal end thereof. The working end portion is an active electrosurgical end capable of supplying electrosurgical currents when the electrode is operatively connected to an electrosurgical apparatus and the later is activated. An opposite proximal end of the body includes a means, such as a connector, for operatively connecting the electrode to the electrosurgical apparatus for supplying the electrosurgical currents to the working end portion.

### TECHNICAL FIELD

The present invention relates to electrosurgical devices and procedures, and more particularly, relates to an electrosurgical electrode having a Freer elevator shape and method for use.

### BACKGROUND

Electrosurgery is a common procedure for dentists, doctors and veterinarians. Electrosurgery involves the application of a high frequency electric current to human (or other animal) tissue as a means to remove lesions, staunch bleeding or to cut tissue with precision. In particular, electrosurgery can be used to cut, coagulate, desiccate or fulgurate tissue. One of the primary benefits of electrosurgery is the ability to make precise cuts with limited blood loss. In electrosurgical procedures, the tissue is burned by an electric current and the number of potential applications for electrosurgery are widespread. For example, electrosurgery is commonly used for such dermatological procedures, such as the removal of skin tags, removal and destruction of benign skin tumors and the removal of warts. Electrosurgery is often performed using a device called an electrosurgical generator, sometimes referred to as an RF Knife.

Electrosurgical handpieces are commercially available that will accommodate a wide variety of electrode shapes and sizes, such as needles, blades, scalpels, balls and wire loops. In addition, the electrosurgical device can be in the form of a multi-function electrode. Many times, the electrosurgical device includes a feature that is attached to a suction source and another feature that permits the device to be attached to a fluid source to permit delivery of the fluid to a location, such as the tip of the device. One technique that is used with the electrosurgical device is the use of suction to capture smoke and plume that is generated during the electrosurgical procedure. Electrosurgical procedures involving tissue excision invariably result in the generation of smoke and odors. This causes several problems including that the smoke interferes with the vision of the surgeon and the smoke can be inhaled by the surgeon and the patient.

In addition, it is often times desirable to deliver fluid, such as an irrigation fluid, to the surgical site at the same as the tissue is cut using the electrosurgical device. Similar to the application of suction to the surgical site, the fluid can be delivered through a conduit that is part of the electrosurgical device or can be another instrument that is used in combination with the electrosurgical device.

Electrocoagulation is a frequent method being used today to achieve hemostasis. However, the conventional devices being used for this purpose have their own limitations including that the design of the device may not be particularly adapted for certain surgical procedures and also, may not perform certain other procedures, such as dissecting or cutting.

In a number of surgical procedures, it is desirable not only to create an incision but also be able to manipulate certain tissue structures after the incision has been made. Typically, in this instance, after the incision is made with one instrument, such as a scalpel or the like, another instrument is inserted through the incision and into place with one tissue structure and then the instrument is manipulated so as to move the tissue structure in a desired manner. For example, when it is desired for a tissue structure to be elevated relative to surrounding structures so as to permit additional surgical procedures, such as a plication of tissue, to be performed, a Freer elevator instrument often can be used. A conventional Freer elevator instrument is an elongated non-energized probe that has an angled end that is configured to cradle the structure (e.g., tissue) that is to be elevated relative to the surrounding tissue. Some Freer elevator instruments are double ended in that each end of the instrument has an angled end for cradling tissue and the like.

It would be desirable to provide an instrument that permits the number of instruments used in a surgical procedure to be reduced and also permits several surgical techniques, such as cutting, coagulation, dissecting, etc., to be performed by a single instrument.

### SUMMARY

An electrosurgical electrode for use in a surgical procedure includes an elongated body in the shape of a Freer elevator (probe) having a working end portion in the shape of a spoon at a distal end thereof. The working end portion is an active electrosurgical end capable of supplying electrosurgical currents when the electrode is operatively connected to an electrosurgical apparatus and the later is activated. An opposite proximal end of the body includes a means, such as a connector, for operatively connecting the electrode to the electrosurgical apparatus for supplying the electrosurgical currents to the working end portion.

The RF energized Freer elevator electrodes disclosed herein provide a number of advantages over conventional instruments, including conventional Freer elevators (non-energized), since the RF energized Freer elevator electrodes are highly useful for cutting, performing coagulation, and dissecting with a high degree of precision. In addition, use of the RF energized Freer elevator electrode permits the tissue to have a low thermal temperature during the performance of the surgical procedure and also there is reduced tissue alteration. The RF energized Freer elevator electrode also provides reduced pain for the patient, faster healing, less swelling and bruising that result in better quality healing.

The electrosurgical electrodes of the present invention have widespread use and can generally be used in any surgical procedure where conventional non-energized Freer elevators where used. For example, the electrosurgical electrodes can be used in ophthalmic facial/orbital reconstruction surgery, including in eye socket contraction, DCR (dacryocystorhinostomy), evisceration, cavernous hemangiomas of the orbit, anophhthalmic socket surgery, etc. Alternatively, the electrosurgical electrode can be used in a surgical procedure to correct penil chordee.

Accordingly, another aspect of the present invention includes performing a surgical procedure that includes the steps of: providing the electrosurgical electrode in the form of a Freer elevator probe having a working end portion in the shape of a spoon at a distal end thereof and at an opposite end an electrosurgical connector capable of supplying electrosurgical currents to tissue when the electrode is connected to an electrosurgical apparatus and the later is activated; activating the electrosurgical apparatus and making an incision in the tissue by means of the electrosurgical currents supplied to the working end portion of the electrode; and deactivating the electrosurgical apparatus and isolating a first tissue structure from a second tissue structure by placing an upper surface of the working end of the electrode underneath the first tissue structure and manipulating the electrode so as to lift the first tissue structure from the second tissue structure with the working end portion. Depending upon the specific surgical procedure being formed, the electrosurgical electrode is then used to perform additional procedures, including applying suction to the surgical site, coagulating blood vessels, etc.

Other features and advantages of the present invention will be apparent from the following detailed description when read in conjunction with the accompanying drawings.

### BRIEF DESCRIPTION OF THE DRAWING FIGURES

The foregoing and other features of the present invention will be more readily apparent from the following detailed description and drawings figures of illustrative embodiments of the invention in which:

FIG. 1 is a perspective view of an electrosurgical electrode according to a first embodiment of the present invention;

FIG. 2 is a side elevation view of the electrosurgical electrode of FIG. 1;

FIG. 3 is a top plan view of the electrosurgical electrode of FIG. 1;

FIG. 4 is a perspective view of an electrosurgical electrode according to a second embodiment of the present invention;

FIG. 5 is a side elevation view of the electrosurgical electrode of FIG. 4;

FIG. 6 is a perspective view of an electrosurgical electrode according to a third embodiment of the present invention;

FIG. 7 is a top plan view of the electrosurgical electrode of FIG. 6;

FIG. 8 is a cross-sectional view taken along the line 8-8 of FIG. 7;

FIG. 9 is a perspective view of an electrosurgical electrode according to a fourth embodiment of the present invention; and

FIG. 10 is a side elevation of the electrosurgical electrode of FIG. 9.

### DETAILED DESCRIPTION OF PREFERRED EMBODIMENTS

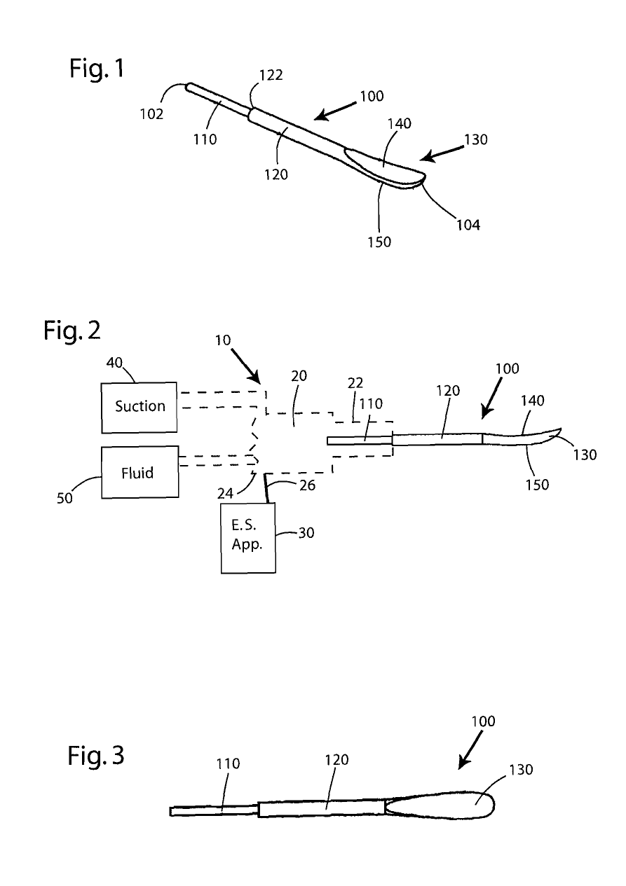

Referring now to FIGS. 1-3, an electrosurgical device 100 according to a first embodiment for use in a surgical procedure is illustrated. The device 100 is in the form of an electrosurgical electrode (e.g., a unipolar electrode) that is part of an electrosurgical system 10 (FIG. 2) and is adapted to be attached to a handpiece 20, such as a handpiece that is described in U.S. Pat. No. 6,001,077 or U.S. Pat. No. 5,196,007, each of which is hereby incorporated by referenced in its entirety. The electrosurgical electrode 100 can be made of any electrically-conductive material, preferably metal, e.g., stainless steel. It is completely coated with an electrically-insulating material (e.g., plastic), except for the distal working end (explained below), and thus is configured to be handled by the surgeon.

The electrosurgical system 10, including the handpiece 20 thereof, is constructed to controllably supply electrosurgical currents to the electrode 100. The electrode 100 can be configured to be attached a nosepiece 22 of the handpiece 20 (which is described in the '007 patent). The handpiece 20 includes a handle 24 having at its side a cable 26 connected at its opposite end to a connector (not shown) for plugging into a standard electrosurgical apparatus 30 that supplies electrosurgical currents to the electrode 100 in a controlled manner. It will also be understood that other handpieces can be used besides those disclosed in the above reference patents so long as the handpieces are part of an electrosurgical system that performs the desired function of delivering an electrosurgical current to the electrosurgical device 100.

The electrosurgical device 100 is operatively connected to an ultra high frequency (RF) radiosurgical energy source (electrosurgical apparatus 30) which operates, in one embodiment, at a frequency of at least 3.0 MHz and preferably within a range from about 3.8 to 4.0 MHz. Studies have shown that the 3.8 to 4.0 MHz frequency range is one exemplary range of RF energy to incise and coagulate tissue because tissue thermal necrosis is minimal and, when interfaced with the apparatus 100, provides excellent cutting of tissue and hemostasis. However, it will be appreciated that the energy source and the electrode 100 can operate in other ranges besides the range that is recited above and different surgical procedures may require some fine tuning or modification of the frequency of the electrode 100. An example of a suitable electrosurgical apparatus 30 is the model SURGITRON Dual-Frequency electrosurgical unit manufactured by and available from Ellman International, Inc. of Oceanside, N.Y.

It is common for the handpiece 20 to have switches (not shown) for remote operation of the electrosurgical apparatus 30. Also shown in FIG. 1 are sources of suction 40 and fluid 50 which may be selectively connected to the hollow handpiece 20 to supply suction or fluid, respectively, to the electrode 100.

The electrode 100 can be thought of as an RF elevator probe in that it is adapted, in part, to function in a manner similar to a Freer elevator. A Freer elevator is a common instrument (probe) in select surgical procedures, such as opthamalogical procedures, nasal procedures, dental procedures, etc. The Freer elevator is conveniently shaped for blunt dissection and tissue manipulation in small spaces. This type of elevator is often used with a small ball of bone wax on its tip to aide in bone hemostasis in hard to reach areas.

As shown in FIGS. 1-3, the electrosurgical electrode or RF energized Freer elevator 100 is an elongated instrument that has a first end 102 and an opposite second end 104. The first end 102 includes a shank portion 110 that has a first dimension, e.g., diameter. The shank portion 110 transitions into a main body portion 120 of the elevator probe 100 and the main body portion 120 has a second dimension, e.g., diameter. The main portion 120 can also be thought of as being a straight body portion of the probe 100. In the illustrated embodiment, the second dimension is greater than the first dimension so as to form a shoulder 122 between the shank portion 110 and the main body portion 120. The shank portion 110 and main portion 120 can have a cylindrical shape, ovoid shape or another shape, such as an irregular shape. The second end 104 includes a working end portion 130 that terminates at the second end 104. The working end portion 130 includes a top surface 140 and an opposing bottom surface 150. The top surface 140 has a contoured shape or configuration that is adapted to perform the intended function of the probe 100 (e.g., isolation of one tissue structure relative to another one).

More specifically, the working end portion 130 has a spoon-like shape in that the top surface 140 has a concave shape and the bottom surface 150 can have a portion 152 that has a convex shape. The shaped working end portion 130 has a slightly upward curvature as shown in FIG. 1. In addition, the curved spoon shape of the working end portion 130 has a wide profile in that, as best shown in FIG. 3, the working end portion 130 slightly tapers outward in its middle portion before tapering back inward toward and terminating with the second end 104. The second end 104 is defined by a curved (arcuate) edge. The curved edge can be a smooth edge.

The electrode 100 can be formed of any number of different conductive materials and in one embodiment, the electrode 100 is formed of stainless steel. It will also be appreciated that the electrode 100 can be formed of two or more different materials. According to one embodiment, the shank portion 110 and main body portion 120 are formed of a first material and the working end portion 130 is formed of a second material. For example, the shank portion 110 and main portion 120 can be formed of stainless steel and the working end portion 130 can be formed of a metal alloy.

In one embodiment, the length of the shank portion 110 is about 0.69 inch and the length of the main portion 120 from the shoulder 122 to the working end portion 130 is about 1.00 inch and the length of the angled working end portion 130 is about 0.85 inch. The width of the probe, in one embodiment, can be about 4 mm.

According to one embodiment, the handpiece 20 includes the nosepiece 22 that receives the electrode 100 (elevator probe). Inside the handpiece 20, a collet can be provided for receiving the electrically conductive shank portion 110 of the electrode 100 for holding the electrode 100 within the handpiece 20. The cable 26 can be electrically connected to the collet which in turn is electrically connected to the electrode 100 so that when the electrosurgical apparatus 30 is turned on, electrosurgical currents are supplied to the electrode 100.

Any number of different coupling techniques can be used for attaching the electrode 100 to the handpiece 20. For example, a male/female connection can be formed between the electrode 100 and the handpiece 20. The shank portion 110 of the electrode 100 can include a hollow bore that receives a member of the handpiece 20 so as to securely couple the two structures to one another. Alternatively, the handpiece 20 can include a hollow bore formed in the nosepiece that receives the shank portion 110 so as to securely couple the two structures to one another.

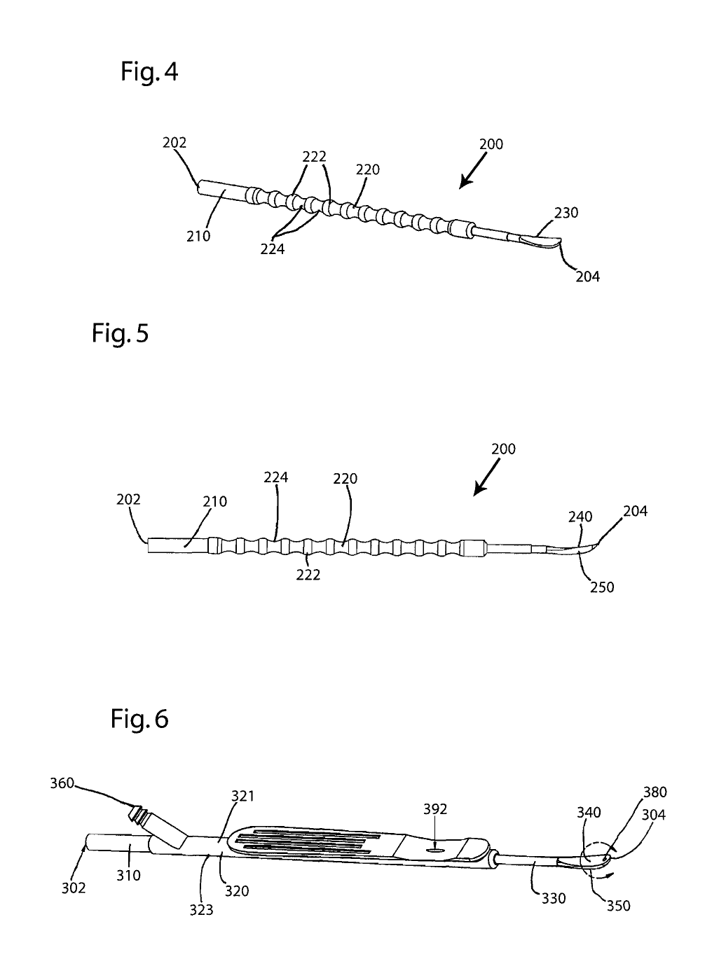

Now referring to FIGS. 4-5, an electrosurgical electrode or RF energized Freer elevator 200 according to a second embodiment is shown. The electrode 200 has some similar features as the electrode 100 and therefore, like elements are numbered alike in the drawings. The electrode 200 is an elongated instrument that has a first end 202 and an opposite second end 204. The electrode 200 has a shank portion 210 that is formed at and terminates with the first end 202; a main body portion 220 and a working end portion 230 formed at and terminating with the second end 204.

The shank portion 210 can be a hollow portion that has a bore 212 formed therein for receiving an object (e.g., handpiece 20) for coupling the electrode 200 to the other object. In other words, the shank portion 210 can include a conventional mono-planar RF connection for coupling the electrode 200 to the energy source. The shank portion 210 can have any number of different shapes, such as cylindrical, ovoid, or other shapes, including irregular shapes.

The main body portion 220 is formed between the shank portion 210 and the working end portion 230 and has a length that is greater than the lengths of each of the shank portion 210 and the working end portion 230. The main body portion 220 includes a series of ribs 222 formed along and spaced apart from one another along the length of the main portion. In the illustrated embodiment, the ribs 222 are in the form of rings that define the areas where the width (diameter) of the main body portion 220 is at its greatest. Accordingly, areas 224 between the ribs 222 have an inward taper and define areas where the width (diameter) of the main body portion 220 is at its minimum.

The main body portion 220 can be formed of an electrically insulating material that permits the probe to be held along this portion 220 and further, it will be appreciated that the main body portion 220 can function as a locating means in that the ribs 222 can have associated indicia (gradations), that are measured from the working end portion and thus, allow the surgeon to generally judge how deep the probe is inserted into an object, such as a body.

At an end opposite where the main body portion 220 interfaces with the shank portion 210, the main body portion 220 interfaces with the working end portion 230. The working end portion 230 is similar to the working end portion 130 of the embodiment of FIG. 1. In other words, the working end portion 230 includes a top surface 240 and an opposing bottom surface 250. The top surface 240 has a contoured shape or configuration that is adapted to perform the intended function of the electrode (probe) 200.

More specifically, the working end portion 230 has a spoon-like shape in that top surface 240 has a concave shape and the bottom surface 250 has a portion 252 that has a convex shape. The shaped working end portion 230 has a slightly upward curvature as shown in FIG. 5. In addition, the curved spoon shape of the working end portion 230 has a wide profile in that, as shown in FIG. 4, the working end portion 230 slightly tapers outward in its middle portion before tapering back inward toward and terminating with the second end 204. The second end 204 is defined by a curved (arcuate) edge. The curved edge can be a smooth edge.

In accordance with one aspect, the shank portion 210 and the main body portion 220 are coated with a material, while the working end portion 230 is left uncovered. For example, the shank portion 210 and the main body portion 220 can be covered with a blue nylon coating. The main body portion 220 of the electrode 200 is the region which a user typically grasps as the surgical procedure is being performed with the electrode 200.

Now referring to FIGS. 6-8, an electrosurgical electrode or RF energized Freer elevator 300 according to a third embodiment is shown. The electrode 300 has some similar features as the electrodes 100 and 200 and therefore, like elements are numbered alike in the drawings. The electrode 300 is an elongated instrument (probe) that has a first end 302 and an opposite second end 304. The electrode 300 has a shank portion 310 that is formed at and terminates with the first end 302; a main body portion 320 and a working end portion 330 formed at and terminating with the second end 304.

The shank portion 310 can be a hollow portion that has a bore 312 formed therein for receiving an object for coupling the electrode 300 to the other object. In other words, the shank portion 310 can include a conventional mono-planar RF connection for coupling the electrode 300 to the energy source. The shank portion 310 can have any number of different shapes, such as cylindrical, ovoid, or other shapes, including irregular shapes.

The main body portion 320 is formed between the shank portion 310 and the working end portion 330 and has a length that is greater than the lengths of each of the shank portion 310 and the working end portion 330. The interface between the main body portion 320 and the shank portion 310 can be defined by a beveled edge, such as a 45 degree chamfer, as opposed to a hard right angle shoulder being formed therebetween. At an end opposite where the main body portion 320 interfaces with the shank portion 310, the main body portion 320 interfaces with the working end portion 330. The working end portion 330 is similar to the working end portion 130 of the embodiment of FIG. 1. In other words, the working end portion 330 includes a top surface 340 and an opposing bottom surface 350. The top surface 340 has a contoured shape or configuration that is adapted to perform the intended function of the electrode (probe) 300.

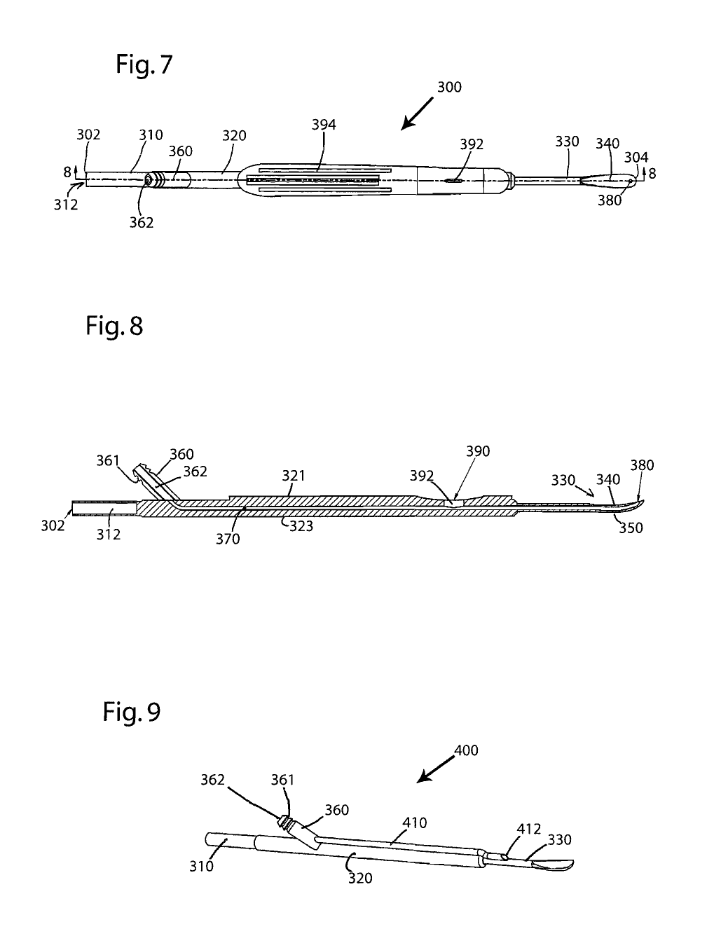

More specifically, the working end portion 330 has a spoon-like shape in that top surface 340 has a concave shape and the bottom surface 350 has a portion 352 that has a convex shape. The shaped working end portion 330 has a slightly upward curvature as shown in FIG. 8. In addition, the curved spoon shape of the working end portion 330 has a wide profile in that, as shown in FIG. 7, the working end portion 330 slightly tapers outward in its middle portion before tapering back inward toward and terminating with the second end 304. The second end 304 is defined by a curved (arcuate) edge. The curved edge can be a smooth edge.

In this embodiment, the electrode (elevator probe) 300 has a feature incorporated therein that permits the probe 300 to be directly connected to a negative pressure (suction) source. More specifically, the main body portion 320 and the working end portion 330 include a suction architecture formed therein to permit the probe 300 to be connected to the suction source so that there is a region of negative pressure along the probe 100 at or near the second end 304 (working end portion 330). The main body portion 320 of the probe 300 has a top surface or edge 321 and an opposite bottom surface or edge 323.

The probe 300 includes a connector 360 formed along the top surface 321 of the main body portion 320 and extending radially outward therefrom. The connector 360 is a hollow body connector in that it includes a channel or bore 362 formed therein. An outer surface of the connector 360 is contoured to permit coupling of a suction conduit, such as suction tubing, to the connector 360. In particular, the outer surface of the connector 360 can have ribs or teeth 361 to create a secure engagement between an end of the suction conduit and the connector 360 by slipping the end of the suction conduit over the connector 360.

The suction architecture of the main body portion 320 includes a main suction bore, conduit or channel 370 that is formed within the main body portion 320 and is in fluid communication at one end with the bore 362. The main suction channel 370 extends the length of the main body portion 320 from the connector 360 and is formed within the working end portion 330 such that the channel 370 extends along the length of the working end portion 330 to a location near or at the second end 304 of the probe 300. More specifically, the channel 370 terminates in the working end portion 330 near the second end 304 with a suction port 380 that is formed along the top surface 340 as shown in FIG. 6. The port 380 can be in the form of a small opening formed along the top surface 340 near the second end 304. Since the channel 370 is directly coupled to the connector 360, negative pressure (suction) can be created at the location of the port 380 by operating the apparatus that creates negative pressure (e.g., motorized pump). In the manner, negative pressure is drawn through the main channel 370 and a material, such as smoke, airborne contaminants, tissue debris, fluid, etc., is captured close to their point of origin, and avoids the need of an additional staff member to hold a separate plume capture device near the excision site. The captured material, such as smoke, is drawn (aspirated) through the port 380 along the main channel 370 and through the connector 360 into the connected suction conduit where it is then delivered to a collection member. As shown in the cross-sectional view of FIG. 8, the main channel 370 is not a completely linear channel in that it includes a first curved section that is in communication with the channel 362 and a second curved section that extends upward to the top surface 340 and terminates with the port 380.

The suction architecture of the probe 300 also includes a suction control member 390 that is formed in the main body portion 320. The suction control member 390 permits the user to selectively control the application of negative pressure to the port 380 and in particular, the suction control member 390 permits the user to selectively release or reduce the strength of the negative pressure. The suction control member 390 can be in the form of an opening 392 that is formed along the top surface 321 of the main body portion 320. In the illustrated embodiment, the opening 392 has an elongated, thin oblong shape (e.g., eye shaped). The suction control member 390 extends downward into the main body portion 320 until it opens into and is in fluid communication with the main channel 370. Since the opening 392 is open at one end to atmospheric conditions and is open at the other end to the main channel 370, the suction control member 390 can act as a means for venting the main channel 370 by introducing atmospheric air into the main channel 370. When the opening 392 is open, the strength of the vacuum will be substantially reduced or eliminated and thus, aspiration at the port 380 is reduced or eliminated.

Conversely, when the user wishes to have probe 300 operate at full vacuum strength, the user simply places a finger over the opening 392 so as to close the opening 392 and thereby prevent atmospheric air from entering the main channel 370. It will further be appreciated that if the user places a portion of his or her finger over the opening 392 so as to only partially cover the opening 392, the strength of the vacuum is only partially reduced and therefore, depending upon how much of the opening 392 is covered, the user can tailor the strength of the vacuum that is applied at the port 380. The top surface 321 of the main body portion 320 can include gripping features, generally identified at 394, that assist in the user holding the probe 300. The gripping features 394 can be in the form of a series of strips that have a roughened outer surface that promotes gripping of the probe 300 during the surgical procedure.

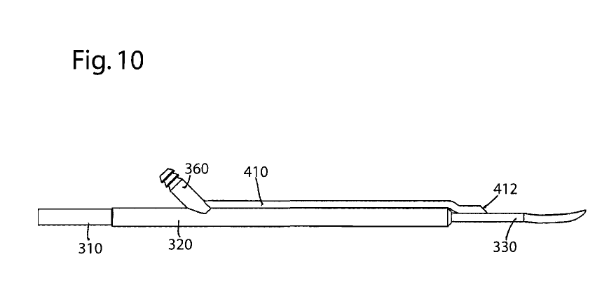

Now referring to FIGS. 9-10, a probe 400 according to another embodiment is illustrated. The probe 400 is similar to the probe 300 in that it includes a fluid conduit that is incorporated therein either for applying a vacuum (negative pressure) to a location along the probe 400 or for delivering a fluid to a location along the probe 400.

The probe 400 includes the shank portion 310; main body portion 320 and working end portion 330; however, unlike the probe 300, the probe 400 does not have a suction architecture incorporated into the main body portion 320 itself. In this embodiment, the channel 362 of the connector 360 does not extend inward into the main body portion 320, but instead, the channel 362 is in fluid communication with an elongated conduit member 410 that is integrally attached to and extends along a length of the main body portion 320. The conduit member 410 is a hollow, tubular like structure that extends along the top surface 321 of the main body portion 320 and also extends along at least a portion of the top surface of the working end portion 330. The interface between the main body portion 320 and the working end portion 330 is preferably defined by a tapered or chamfered surface (e.g., a 45 degree angled surface) and therefore, the conduit member 410 also has a beveled section 420 at the interface between the main conduit member 320 and the working end portion 330.

A free distal end 412 of the conduit member 410 is located along the top surface of the working end portion 330 prior to the spoon-like construction thereof. The distal end 412 can be defined by a beveled edge, such as a 45 degree edge.

When it is desired for the conduit member 410 to act as a suction source, a vacuum conduit is connected to the connector 360 in a manner previously described in greater detail and then the vacuum source is operated so as to create negative pressure within the conduit member 410 and at the free distal end 412. The application of negative pressure at the distal end 412 causes any fluid or material located proximate thereto (e.g., fluid and material that is located within or proximate the spoon-like portion of the working end portion 330) to be drawn into the conduit member 410 and then delivered to a collection member.

When it is desired for the conduit member 410 to act as a fluid delivery system, a source of fluid is connected to the connector 360 using a conduit member, such as flexible tubing. A pump or the like is activated to cause fluid to be delivered to the connector 360, through the channel 362 and into the conduit member 410 where it flows the length thereof and is discharged through the open distal end 412 of the conduit member 410. In this manner, the fluid is delivered to the spoon-like portion of the working end portion 330 of the probe 400. The fluid can be an irrigation fluid or the like.

As a result of the relatively simple construction, manufacture of the RF energized Freer elevators of the present invention is quite simple and has a low associated cost, which is important for different settings, including where the probes are for use in a disposable hospital environment.

When RF energy is supplied, it flows to the working end of the probe and allows for dissection and excision of different types of tissue and other structures, while at the same time effectively coagulating any bleeders that may result.

Each of the RF Freer elevator probes disclosed herein enables the surgeon to provide the necessary surgical features of cutting, coagulation and suction, with or without suction or fluids, with RF energy being applied during part or all of the time that the dissection procedure is carried out, with RF energy and blunt dissection, or with blunt dissection, or with suction along without RF energy being applied. The surgeon would be otherwise required to utilize several different surgical instruments to accomplish what the RF Freer elevator probe alone can accomplish. The changing of instruments during the surgical intervention prolongs the surgery, blood loss and anesthetic time for the patient.

By interfacing the RF energized probe with the ultra-high 3.8-4.0 MHz radiosurgery apparatus (apparatus 30), a number of surgical and clinical advantages are realized including but not limited to: better operative results, due to the high frequency radiosurgery device's ability to significantly reduce tissue necrosis, minimal scarring, reduced surgical pain and post-operative pain, and controlled bleeding and post-operative bleeding.

Precise 3.8-4.0 MHz high frequency/low temperature dissection, using the RF energized monopolar (unipolar) Freer elevator probe to cut and coagulate bleeding vessels under direct vision, as well as the lifting characteristics and form of the working end of the Freer elevator probe permits precise surgical procedures to be performed. The radiofrequency method drastically reduces the risk of complications (bleeding, infection, asymmetry, etc.). Additionally, there is typically a shorter recovery period.

### EXAMPLES

In accordance with the present invention, one of the electrosurgical electrodes (probes) 100, 200, 300, 400 is used in an electrosurgical apparatus that is used in ophthalmic facial/orbital reconstruction surgery. For example, the elevator probes 100, 200, 300, 400 can be used in eye socket contraction, DCR (dacryocystorhinostomy), evisceration, cavernous hemangiomas of the orbit, anophhthalmic socket surgery, etc.

For example, when an individual develops tearing due to acquired obstruction of the nasolacrimal (tear) duct, a DCR procedure is usually offered to the individual as a means for correcting the condition. Lacrimal drainage surgery is called dacryocystorhinostomy (DCR) and can be performed in different ways. One type of operation is an external DCR where an incision is made on the side of the nose, where eyeglasses might rest. A small amount of bone is removed to permit a new connection between the lacrimal sac and the inside of the nose. Small plastic tubes are inserted at the time of surgery to keep the newly created opening from scarring shut during the healing process. The tubing is removed a few months after surgery.

It will therefore be appreciated that in the above surgical procedures, the RF elevator probes 100, 200, 300, 400 are conveniently shaped for blunt dissection and tissue manipulation in small spaces and therefore, they find particular utility in the above surgical procedures

It will also be understood that the present examples are merely exemplary of a number of different surgical procedures where the RF elevator probes 100, 200, 300, 400 (electrosurgical devices) according to the present invention can be used; however, the above list is merely exemplary and not limiting of the scope of the applications in which the present electrosurgical devices can be used. The electrosurgical devices (RF elevator probes) of the present invention are capable of being used in any surgical procedure where a classical Freer elevator can be used and further, the electrosurgical aspect thereof allows the RF energized RF elevator probes to offer significant surgical benefits and the same useful clinical and patient advantages still apply.

### Example

Penile chordee, with and without hypospadias, is amenable to surgical correction. The term “penile chordee” refers to a condition where there is ventral bending of the penile shaft. Hypospadias is the most common congenital anomaly of the penis and refers to an abnormal penile configuration in which the urethral meatus is located on the ventral surface of the penis, proximal to the end of the glands, and anywhere from the ventral gland to the perineum. Epispadias refers to the condition in which the meatus is located on the dorsal surface of the penis. Penile chordee is often associated with hypospadias, and may be due to tethering or dysplasia of the ventral penile shaft skin. A dorsal hood of incomplete prepuce may also be present. There is no single known cause of hypospadias; however, genetic factors exist, most likely based on a multifactorial mode of inheritance.

The more common surgical procedures to correct penile chordee are variations of the Nesbit plication technique which involves a dorsal plication of the ventral tunica albuginea and is effective in most cases of corporal disproportion. The corpus cavernosum penis is one of a pair of a sponge-like regions of erectile tissue which contain most of the blood in the male penis during erection. The corpus cavernosum and corpus spongiosum (also known as the corpus cavernosum urethra) are three expandable erectile tissues along the length of the penis which fill with blood during erection. The two corpora cavernosa lie along the penis shaft, from the pubic bones to the head of the penis, where they join. These formations are made of a sponge-like tissue containing irregular blood-filled spaces lined by endothelium and separated by connective tissue septa. The corpus spongiosum is one smaller region along the bottom of the penis, which contains the urethra and forms the glands penis

Plicating the dorsum of the corpora shortens that aspect of the penis to correct curvature. Some surgeons incise directly through the Buck's fascia (a layer of deep fascia covering the penis) to place the plicating sutures. This approach risks inadvertent injury to the neurovascular bundles, which are located on either side of the dorsal midline with branches ramifying distally around the corpora cavernose to the ventral side of the phallus. When the corpora cavernosa are plicated, the Buck's fascia is elevated with its encased neurovascular bundle in a manner that attempts to avoid any direct injury to these nerves. A hazard with this approach is therefore the potential inclusion of the dorsal neurovascular bundle with the resultant erectile and sensory dysfunction. Other possible hazards of the technique are that the incision through the tunica albuginea may enter the erectile tissue and adversely affect its function.

Other surgical procedures exist for correcting penile chordee including a technique to increase the length of the short or ventral aspect of the corpora cavernosa as an alternative to plicating or shortening the long side of the curved penis. However, these other surgical procedures have disadvantages associated therewith and therefore, there is a desired need for a surgical procedure for correcting penile chordee that ensures the no injury occurs to the neurovascular bundle during the plication step.

In accordance with the present invention, one of the electrodes (probes) 100, 200, 300, 400 is used in an electrosurgical apparatus that is used in a surgical procedure for correcting penile chordee. The elevator probes 100, 200, 300, 400 are conveniently shaped for blunt dissection and tissue manipulation in small spaces. According to the surgical procedure of the present invention, one of the elevator probes 100, 200, 300, 400 is used to isolate the neurovascular bundle during the plication step of the surgical procedure.

According to the surgical procedure, the penis is degloved using standard surgical procedures and then an incision is made through Buck's fascia. In particular, the incision through Buck's fascia is made lateral and parallel to the neurovascular bundle at the maximum level of the chordee. A similar incision is carried out on the contralateral side, separating Buck's fascia and underlying layers from the tunica albuginea. Following isolation of the neurovascular bundle, each corporal body is plicated by creating a longitudinal incision through the tunica albuginea, which is then closed transversely with a suture, such as a 5-0 polydioxanone suture. The Buck's fascia is then subsequently closed with an absorbable suture or the like following confirmation of the chordee correction.

Applicants have found that a study group of patients undergoing the above procedure experienced no complications and none of these patients required reoperation for persistent chordee. Applicants' present surgical technique involves elevation of the neurovascular bundle prior to plication using one of the probes disclosed herein, thereby ensuring no inadvertent injury to the neurovascular bundle. Utilization of the Freer elevator type device only adds a small amount of time to chordee correction compared to a conventional technique that uses a standard plication lateral to the neurovascular bundles.

The working end portion of any of the disclosed probes can be used with a small ball of bone wax on its tip which aids in bone hemostasis in hard to reach areas.

It will be understood and appreciated that the RF energized Freer elevator probe 100, 200, 300, 400 provides a number of advantages over conventional instruments, including conventional Freer elevators (non-energized), since the RF energized Freer elevator probe is highly useful for cutting, performing coagulation, and dissecting with a high degree of precision. In addition, use of the present RF energized Freer elevator probe permits the tissue to have a low thermal temperature during the performance of the surgical procedure and also there is reduced tissue alteration. The RF energized Freer elevator probe also provides reduced pain for the patient, faster healing, less swelling and bruising that result in better quality healing.

While exemplary drawings and specific embodiments of the present invention have been described and illustrated, it is to be understood that the scope of the present invention is not to be limited to the particular embodiments discussed. Thus, the embodiments shall be regarded as illustrative rather than restrictive, and it should be understood that variations may be made in those embodiments by workers skilled in the art without departing from the scope of the present invention as set forth in the claims that follow, and equivalents thereof. In addition, the features of the different claims set forth below may be combined in various ways in further accordance with the present invention.

What is claimed is:

1. An electrosurgical electrode for use in a surgical procedure comprising:
an elongated body in the shape of a Freer elevator having a working end portion in the shape of a spoon at a distal end thereof, the working end portion being an active electrosurgical end capable of supplying electrosurgical currents when the electrode is operatively connected to an electrosurgical apparatus and the later is activated, wherein an opposite proximal end of the body includes a means for operatively connecting the electrode to the electrosurgical apparatus for supplying the electrosurgical currents to the working end portion.

2. The electrosurgical electrode of claim 1, wherein the working end portion is angled upwardly relative to a main straight portion of the elongated body.

3. The electrosurgical electrode of claim 1, wherein the electrosurgical currents have a frequency of between about 3.8 MHz and about 4 MHz.

4. The electrosurgical electrode of claim 1, wherein the working end portion has a concave shaped upper surface and a rounded distal end so as to form the spoon shape.

5. The electrosurgical electrode of claim 1, further including:
a fluid transfer means associated with the elongated body and including a hollow connector extending outwardly from the elongated body and an internal channel formed within the elongated body, the channel being in communication with the inside of the connector at one end and terminating in a port at second end, the port being an opening formed along a top surface of the spoon shaped working end portion.

6. The electrosurgical electrode of claim 5, wherein the fluid transfer means includes a source of negative pressure that is operatively connected to the connector so as to cause negative pressure within the fluid conduit for aspirating a fluid or material at the working end portion.

7. The electrosurgical electrode of claim 5, wherein the fluid transfer means includes a source of fluid that is operatively connected to the connector so as to deliver fluid into and through the connector and into the main channel where it flows to the open end where it is discharged along the working end portion.

8. The electrosurgical electrode of claim 1, wherein the elongated body has a means for applying suction to the working end portion of the elongated body, the means including a connector that extends outwardly from the elongated body and is adapted to mate with a conduit connected to a suction source and a main channel formed within and along a length of the elongated body and terminating in a port formed along an upper surface of the working end portion proximate a curved distal end thereof.

9. The electrosurgical electrode of claim 8, wherein the connector comprises a stem extending radially outward from the elongated body, the elongated body including a bore formed therethrough that is in fluid communication with one end of the main channel, an opposite end of the main channel including an angled section that extends upward to the top surface of the working end portion where the main channel terminates in the port which is formed along an upper surface of the spoon shaped working end portion.

10. The electrosurgical electrode of claim 1, wherein the electrosurgical electrode comprises an elongated body having a fluid transfer means incorporated therein, the fluid transfer means including a connector that extends outwardly from the elongated body and a fluid conduit that extends along a length of the elongated body and is separate therefrom, the fluid conduit being fluidly connected at one end to the connector and at an opposite end, the fluid conduit terminating in an opening that is located along or proximate the working end portion proximate a distal end of the electrode.

11. The electrosurgical electrode of claim 8, further including a suction control member that permits the user to selectively control the application of suction to the working end portion of the elongated body.

12. The electrosurgical electrode of claim 11, wherein the suction control member comprises an opening formed along the elongated body and in fluid communication with the main channel for selectively venting the main channel to atmosphere.

13. The electrosurgical electrode of claim 12, wherein the opening of the suction control member is formed in a recessed thumb region of a hand grip portion that is formed along a top surface of the elongated body.

14. The electrosurgical electrode of claim 1, wherein the means for operatively connecting the electrode to the electrosurgical apparatus comprises a bore formed in the elongated body at the proximal end that is constructed to receive a complementary nosepiece of the handpiece.

15. The electrosurgical electrode of claim 1, wherein the means for operatively connecting the electrode to the electrosurgical apparatus comprises a shank portion that is constructed to be received within a complementary bore formed in one end of the handpiece.

16. An electrosurgical system comprising:
an electrosurgical electrode in the shape of a Freer elevator having a working end portion in the shape of a spoon at a distal end thereof, the working end portion being an active electrosurgical end capable of supplying electrosurgical currents, wherein an opposite proximal end includes a first connector;
a handpiece having a second connector at one end thereof for mating with the first connector resulting in the electrosurgical electrode being detachably coupled to the handpiece; and
a source of electrosurgical currents, the handpiece being operatively connected to the source of electrosurgical currents so as to controllably deliver the electrosurgical currents to the working end of the electrode when the system is activated.

17. The electrosurgical system of claim 16, wherein the working end portion is angled upwardly relative to a main straight portion of the elongated body.

18. The electrosurgical system of claim 16, wherein the source of electrosurgical currents comprises an electrosurgical apparatus capable of supplying unipolar RF electrosurgical currents at a frequency of about 3.8 MHz to 4.0 MHz.

19. The electrosurgical electrode of claim 16, wherein the electrosurgical currents have a frequency of between about 3.8 MHz and about 4 MHz.

20. The electrosurgical system of claim 16, wherein the working end portion has a concave shaped upper surface and a rounded distal end so as to form the spoon shape.

21. The electrosurgical electrode of claim 16, further including:
a fluid transfer means associated with the elongated body and including a hollow connector extending outwardly from the elongated body and an internal channel formed within the elongated body, the channel being in communication with the inside of the connector at one end and terminating in a port at second end, the port being an opening formed along a top surface of the spoon shaped working end portion.

22. The electrosurgical system of claim 21, wherein the fluid transfer means includes a source of negative pressure that is operatively connected to the connector so as to cause negative pressure within the fluid conduit for aspirating a fluid or material at the working end portion.

23. The electrosurgical system of claim 16, wherein the elongated body has a means for applying suction to the working end portion of the elongated body, the means including a connector that extends outwardly from the elongated body and is adapted to mate with a conduit connected to a suction source and a main channel formed within and along a length of the elongated body and terminating in a port formed along an upper surface of the working end portion proximate a curved distal end thereof.

24. The electrosurgical system of claim 23, wherein the connector comprises a stem extending radially outward from the elongated body, the elongated body including a bore formed therethrough that is in fluid communication with one end of the main channel, an opposite end of the main channel including an angled section that extends upward to the top surface of the working end portion where the main channel terminates in the port which is formed along an upper surface of the spoon shaped working end portion.

25. The electrosurgical system of claim 16, wherein the electrosurgical electrode comprises an elongated body having a fluid transfer means incorporated therein, the fluid transfer means including a connector that extends outwardly from the elongated body and a fluid conduit that extends along a length of the elongated body and is separate therefrom, the fluid conduit being fluidly connected at one end to the connector and at an opposite end, the fluid conduit terminating in an opening that is located along or proximate the working end portion proximate a distal end of the electrode.

26. The electrosurgical system of claim 23, further including a suction control member that permits the user to selectively control the application of suction to the working end portion of the elongated body.

27. A surgical procedure comprising the step of:
providing an electrosurgical electrode in the form of a Freer elevator having a working end portion in the shape of a spoon at a distal end thereof and at an opposite end an electrosurgical connector capable of supplying electrosurgical currents to tissue when the electrode is connected to an electrosurgical apparatus and the later is activated;
activating the electrosurgical apparatus and making an incision in the tissue by means of the electrosurgical currents supplied to the working end portion of the electrode; and
deactivating the electrosurgical apparatus and isolating a first tissue structure from a second tissue structure by placing an upper surface of the working end of the electrode underneath the first tissue structure and manipulating the electrode so as to lift the first tissue structure from the second tissue structure with the working end portion.

28. The surgical procedure of claim 27, wherein the electrosurgical connector is a bare metal end of the probe and further including the step of:
coupling the electrosurgical electrode to a handpiece that is operatively connected to the electrosurgical apparatus.

29. The surgical procedure of claim 27, wherein the step of activating the electrosurgical apparatus comprises supplying unipolar RF electrosurgical currents to the working end portion that have a frequency between about 3.8 MHz to about 4.0 MHz.

30. The surgical procedure of claim 27, wherein the procedure is selected from the group consisting of ophthalmic facial/orbital reconstruction surgery and a procedure for correction of penile chordee.

31. The surgical procedure of claim 30, wherein the step of isolating a first tissue structure from a second tissue structure comprises the steps of isolating the neurovascular bundle during a surgical procedure to correct penile chordee by elevating the neurovascular bundle from surrounding tissue by inserting the spoon shaped working end portion of the electrode underneath the Buck's fascia of the penis after an incision is made through the Buck's fascia with the RF energized working end portion.

32. The surgical procedure of claim 31, wherein the step of inserting the working end portion comprises manipulating the working end portion so as to cause separation of the Buck's fascia from the tunica albuginea of the penis.

33. The surgical procedure of claim 27, wherein the surgical procedure is a dacryocystorhinostomy procedure and includes the steps of: forming an incision in a side of the nose with the working end portion after activating the electrosurgical apparatus and removing an amount of bone.
